(19) United States
(12) Patent Application Publication
(10) Pub. No.: US 2013/0190539 A1
(43) Pub. Date: Jul. 25, 2013

(54) SYSTEM AND METHOD FOR SECONDARY USES OF GLYCOL AND WATER AFTER DEICING

(71) Applicant: HYDRATION SYSTEMS, LLC, Scottsdale, AZ (US)

(72) Inventors: John R. Herron, Corvallis, OR (US); Keith Lampi, Corvallis, OR (US); Sherwin Gormly, Carson City, NV (US); Edward Beaudry, Corvallis, OR (US)

(73) Assignee: HYDRATION SYSTEMS, LLC, Scottsdale, AZ (US)

(21) Appl. No.: 13/746,160

(22) Filed: Jan. 21, 2013

Related U.S. Application Data
(60) Provisional application No. 61/589,118, filed on Jan. 20, 2012.

(51) Int. Cl. C07C 29/86; B01D 61/58

(52) U.S. Cl. CPC C07C 29/86; B01D 61/58
USPC 568/919; 210/195.2

(57) ABSTRACT

A system and method for extracting the glycol from used or spent glycol mixtures, particularly aircraft deicing fluid, and simultaneously using the extracted water to hydrate brines, such as pavement deicing brines, are disclosed. The system provides comprehensive fluid handling and no discharge, as it re-concentrates glycol from spent aircraft deicing fluids.

### CROSS-REFERENCE TO RELATED APPLICATION

This application claims priority to U.S. provisional application Ser. No. 61/589,118 filed Jan. 20, 2012, the entire disclosure of which is hereby incorporated by reference.

### BACKGROUND

Airplane and runway deicing is a critical component in insuring the safety of travelers. Typical deicing fluids may comprise organic and heavy metal contaminants that would otherwise contaminate aircraft runoff water and make it environmentally unsound. The runoff water is therefore not suitable for use in hydrating the pavement deicing fluids/brines prior to spreading, or overall water loss to the ambient environment and watershed.

### SUMMARY OF THE INVENTION

A system and method for extracting the glycol from used or spent glycol mixtures, particularly aircraft deicing fluid, and simultaneously using the extracted water to hydrate brines, such as pavement deicing brines, are disclosed. The system provides comprehensive fluid handling and no discharge, as it re-concentrates glycol from spent aircraft deicing fluids.

### BRIEF DESCRIPTION OF THE DRAWING

FIG. 1 is a schematic illustration of the process and system according to a preferred embodiment of the invention.

### DETAILED DESCRIPTION OF THE INVENTION

### Overview

Implementations of the present disclosure utilize forward osmosis (FO) membranes to recover the clean water component from used or spent glycol mixtures, such as spent aircraft deicing fluid. The recovered clean water component may then be reused to hydrate brines, such as pavement-deicing brine for spreading on adjacent runways and taxiways. In an embodiment of the invention, the FO process reclaims and re-concentrates the glycol component of the aircraft deicing fluid in the liquids rejected by the FO membrane element, and supplies it for beneficial reuse. At substantially the same time, the water that passes through the FO membrane receives reverse osmosis (RO) level water treatment while being passively drawn into the salt that will be used to do perform pavement deicing at the same or other aerospace support facility (airport or air base). In this way, the glycol is recovered and concentrated on one side of the FO membrane for beneficial reuse, and membrane-treated water is drawn into the salt to provide hydration of these pavement deicing fluids. Secondary uses of glycols, such as propylene glycol, are included for anything from high grade applications to low grade applications, such as portable toilets.

The invention involves a process for simultaneously recovering secondary effluent as clean hydrothermal reinsertion water and production of FO “power” to drive the process. Forward osmosis is employed, wherein the systems sets up an osmotic potential difference on either side of the FO membrane, resulting in the simultaneous extraction of glycol from, and treatment of the excess water component in, spent aircraft deicing fluid. The treated water extracted by FO across the FO membrane is used to supply dry salts, which are then used to generate clean pavement deicing brine. The system uses a substantial osmotic power/force/potential difference to create a synergistic energy advantage across a semi-permeable membrane, which in turn provides most if not all the force required to push the water across the membrane, thereby treating it. Accordingly, FO is used as the driving force for the process.

In an embodiment of the invention, the membrane-treated water may comprise up to (and in some cases may exceed) about 98% rejection of all organics and heavy metal contaminants including but not limited to glycols and aluminum/iron salts. Thus, the system of the invention can essentially remove the aforementioned environmental contaminants from the spent deicing fluid (i.e., the aircraft water runoff) while hydrating brines for re-use as deicing agents for other purposes, such as pavement deicing. This has the beneficial effect of preventing the contaminants from being in the deicing agents produced by the system, thus preventing the spreading of these contaminants into the ambient environment and watershed.

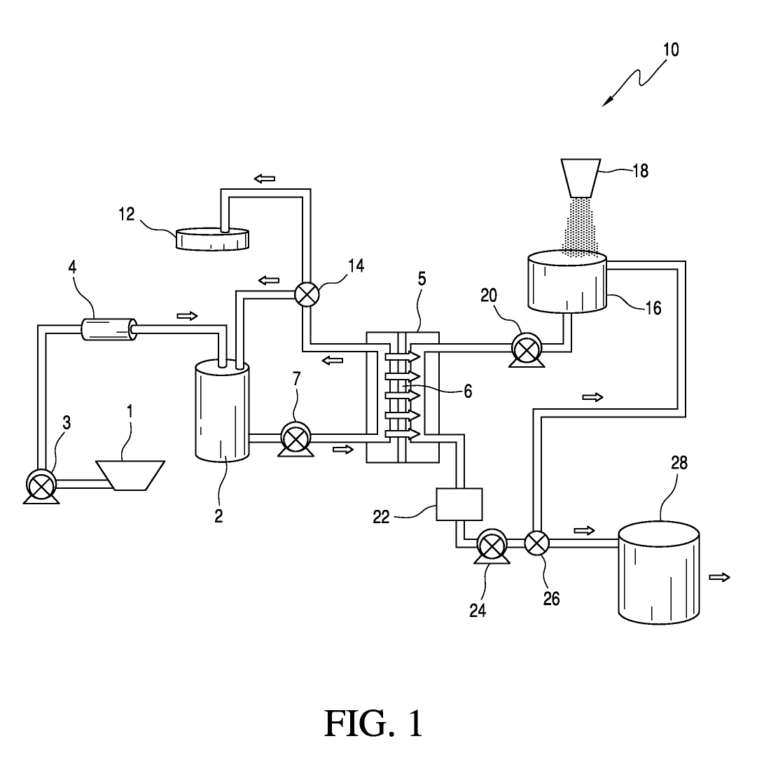

The method of the invention is a synergistic process where the majority of the energy required for the treatment process (i.e., forcing of the water through the selectively permeable membrane) is provided by the osmotic potential of the salt to be hydrated, rather then the use of high-pressure pumps as in RO processes. Various implementations may comprise any low energy, high rejection membrane system. The combined process of glycol recovery with a salt hydration system is shown in FIG. 1.

### System and Processes

FIG. 1 illustrates a schematic illustration of a general description of one exemplary embodiment or implementation of the system and process of the invention. The system 10 shown in FIG. 1 provides for the collection of aircraft deicing fluid in a storage sump 1. In various implementations, this may comprise a tank that may be underground at the end of the collection drain in the aircraft deicing area of the facility, and another tank that may be above ground and located near and pumped from the same or any other tank or receptacle for holding deicing run off. Once this spent deicing and surfaces runoff fluid is delivered to any appropriate closed containment unit (such as a tank or other vessel), the runoff fluid may be delivered to the primary separation (treatment) tank 2. Primary separation tank 2 is a holding tank for the deicing fluid, before water is removed from the deicing fluid by running it through forward osmosis (FO) membrane element 5. In still another exemplary embodiment, delivery to the separation tank to enter the treatment process may be via a sump pump 3, and may optionally pass through a filter 4 or a combination of sump pump and filter. The filter 4 is preferably a rough filter, such as a 50 micro bag filter. The filter 4 removes dirt and suspended solids.

Fluid within the system is transported within the system via pipes or other conduits which link the various components or elements together. The term “connected” as used in the specification and claims means either a direct or indirect connection between two or more conduits, components or elements. Therefore, as used herein, the term “connected” or “connecting” regarding two or more elements or structures is used broadly, to encompass situations in which there may be one or more other elements or structures between the two “connected” elements. For example, as illustrated in FIG. 1, the sump 1 is connected to the filter 4, the connection being via one or more pipes or other conduits though which fluid can travel from sump 1 to filter 4.

Storage and circulation of the glycol containing aircraft deicing fluid and its associated deicing area runoff water may start at this primary separation tank 2 in some implementations. From this tank 2 the runoff is pumped through the glycol recovery loop (shown on the left side of FIG. 1) and the reject side of the FO membrane element 5. Up to about 98% of the water in this loop can be harvested (left to right in FIG. 1) across the FO membrane element 5 into the pavement deicing brine loop (shown on the right side of FIG. 1), depending on the water content/dilution level of the runoff and the desired dilution of the pavement deicing salts. In various implementations, any percentage of the water from the glycol recovery loop may be harvested across an FO membrane element.

A preferred embodiment of the system of the invention generally comprises the following components or elements: a sump for collecting the fluid; a filter connected to the sump; a separation tank connected at its first inlet to the filter, the separation tank having a second inlet and an outlet; a forward osmosis element having a first inlet and a first outlet, and a second inlet and a second outlet, the first inlet of the forward osmosis element connected to the outlet of the separation tank; a reclaimed brine tank connected to the first outlet of the forward osmosis element; a brine loop connecting the first outlet of the forward osmosis element to the second inlet of the forward osmosis element; a first valve disposed between and connected to the brine loop and the reclaimed brine tank; a recovered glycol tank; and a glycol loop connecting the second outlet of the forward osmosis element to the recovered glycol tank and the separation tank, the glycol loop having a second valve disposed between an connected to the glycol tank and the separation tank.

The preferred method according to the invention for reclaiming spent aircraft deicing fluid, generally comprises the following steps: (a) filtering the fluid; (b) subjecting the fluid to forward osmosis in a forward osmosis element, to produce a first stream comprising primarily glycol and a second stream comprising primarily brine; combining a first portion of the first stream produced in step (b) with additional spent aircraft deicing fluid to produce a combined fluid, and subjecting the combined fluid to forward osmosis in the forward osmosis element; (d) recovering a second portion of the first stream produced in step (b) comprising primarily glycol; (e) combining a first portion of the second stream produced in step (b) with salt to produce a brine, and transporting the first portion to the forward osmosis element's permeate side, to drive forward osmosis in steps (b) and (c); and (f) recovering a second portion of the second stream produced in step (b).

The system and process generally work as follows.

Osmotic potential drives the flow or harvest of water through the membrane 6 in the FO membrane element 5 from left to right. (The phrase “left to right” as used herein is not actually limited to left- or right-handedness. Rather, in the particular configuration of an embodiment of the system shown in FIG. 1, wherein fluid from the primary separation tank 2 is transported into the FO membrane element 5 via pump 7, and water from the fluid travels through the membrane to the side of the element 5 to the permeate side of the element 5.) The water from which glycol has been substantially removed then flows to the product brine loop tank 22.

As little as about 2% or less by volume of the runoff that is collected may remain when water removal is complete. In various implementations, the residuals are concentrated up into the reusable glycol solution and sent to the recovered glycol tank 12. This process can be done in either a batch or continuous basis at the glycol side loop recovery valve 14 shown. In most implementations, a batch process may be utilized in order to achieve the highest glycol recovery concentrations.

In a batch process, valve 14 recycles (directs) substantially all the fluid back to tank 2, and the system 10 is run until the concentration in tank 2 reaches the desired level. At that point, valve 14 is redirected to send substantially all fluid in the system to recovered glycol tank 12. The system is then refilled with spent deicing fluid, and the process is repeated.

An embodiment of a batch process according to the invention generally comprises the following steps: (a) filtering the spent deicing fluid; (b) subjecting the fluid to forward osmosis in a forward osmosis element, to produce a first stream comprising primarily glycol and a second stream comprising primarily brine; (c) recovering substantially all of the first stream produced in step (b); (d) combining a first portion of the second stream produced in step (b) with salt to produce a brine, and transporting the first portion to the forward osmosis element's permeate side, to drive forward osmosis in step (b); and (e) recovering a second portion of the second stream produced in step (b).

In contrast to a batch system, in a continuous system, valve 14 recyles (directs) some (but not substantially all) of the fluid flowing to it (from element 5) to tank 12, and valve 14 returns the majority of the fluid flowing to valve 14 (from element 5) to tank 2. The ratio of fluids directed by valve 14 to each of tanks 2 and 12 is selected in order to maintain the concentration of the deicing fluid being sent to tank 12 at the desired level. Optionally, the ratio of fluids being directed by valve 14 to tank 2 and tank 12 may be varied during operation.

An embodiment of a continuous process according to the invention generally involves the following steps: (a) filtering the spent deicing fluid; (b) subjecting the filtered fluid to forward osmosis in a forward osmosis element, to produce a first stream comprising primarily glycol and a second stream comprising primarily brine; (c) combining a first portion of the first stream produced in step (b) with additional spent aircraft deicing fluid to produce a combined fluid, and subjecting the combined fluid to forward osmosis in the forward osmosis element; (d) recovering a second portion of the first stream produced in step (b) comprising primarily glycol; (e) combining a first portion of the second stream produced in step (b) with salt to produce a brine, and transporting the first portion to the forward osmosis element's permeate side, to drive forward osmosis in steps (b) and (c); and (f) recovering a second portion of the second stream produced in step (b).

In some implementations, dry salt may be added to the pavement salt dissolving mix tank 16 (shown in the upper right in FIG. 1). In FIG. 1, a receptacle 18, such as a hopper, is illustrated for storing and/or dispensing salt into the tank 16. In general, the greater the amount of salt, the faster the water will be drawn across the FO membrane 6 in the FO membrane element 5. Accordingly, a user may adjust salt levels to control the rate or timing of the system. A small amount of dilute pavement deicing fluid may be present in the salt dissolving mix tank 16 at a ratio at or just below that required to provide saturation concentration for the dry salt being added. At least a portion of the resulting near saturation salt brine may then be pumped to the permeate (water producing) side (shown on the right side of the FO in FIG. 1) of the FO membrane element 5. Pumping of the brine may be assisted by a pump 20. Here, the osmotic potential of the brine draws clean water from the glycol recovery loop through the membrane to provide the desired pavement deicing fluid concentration. The clean, balanced product brine may then flow to the product brine tank 22 in some implementations.

Optionally, the system of the invention is supplied with appropriate sensors and controls to regulate the water recovery rate while maintaining product specifications, i.e., for pavement deicing salt brine concentration specifications. Thus, the process may optionally involve using an electro-conductivity (EC) sensor and control to monitor salt input and/or finished brine recycle rate.

The reclaimed brine, also referred to as the balanced pavement deicing brine, may be transported (such as via pump 24) from the product brine tank 22 to a three-way valve 26 that is used in an embodiment of the invention. The three-way valve 26 permits the system's operator to control the salt addition rate, according to the strength requirements of the product pavement deicing brine. The three-way valve 26 may then supply at least a small percentage or portion of this dilute brine back to the salt dissolving mix tank 16. A majority of the dilute brine may be sent to the reclaimed brine tank, also referred to as the pavement deicing brine facility supply tank 28. In various implementations, any percentage or portion of dilute brine may be sent back to the salt dissolving mix tank 16, and any percentage or portion may be sent to the pavement deicing brine facility supply tank 28. In most implementations, the percentage of brine required for recycle back to the brine tank 16 will be small in comparison to the product brine when the system is operating at maximum production capacity.

Optionally, a higher brine recirculation rate (percentage by volume) and a slower dry salt addition rate may be used in some implementations to lower the osmotic potential at the FO membrane. This will subsequently moderate the water recovery rate of the system 10, if this is desired to balance input fluid recovery to output fluid demand.

As a restatement of or in addition to what has been already been described and disclosed above, the system 10 may comprise at least one FO system 5. The FO system 5 may be comprised of any FO membrane configured to allow water to pass from a first loop to a second loop. In an implementation, the first loop may comprise a glycol loop and the second loop may comprise a brine loop.

The glycol loop may be comprised of any variety of combinations of tanks, pumps, and valves that allow a glycol solution to be moved to a FO system. In an implementation, the glycol loop may comprise a primary separation tank 2. After progressing through the primary separation tank, the glycol solution may travel to the FO. Any solution or product remaining after the FO system may then travel to a glycol loop recovery valve 14. The glycol loop recovery valve 14 may then be configured to send product to either or both the primary separation tank 2 and/or a recovered glycol tank 12.

Optionally, glycol concentration at different places in the system can be measured and monitored. Examples of monitoring options are previously calibrated in-line turbidity measuring devices and/or volumetric flow meters. These monitoring devices can be coordinated through programmable logical controls.

Prior to entering the glycol loop, deicing fluid or any other fluid may be collected in a tank or a sump 1. The deicing fluid may pass through a filter 4 (for example, a micron filter) before entering the primary separation tank 12. In other implementations, any variety of filters, tanks, sumps, and the like may be utilized.

The product brine loop may comprise any variety of combinations of tanks, pumps, and valves that allow a nearly saturated salt solution to be moved through the FO system 5. In an implementation, the product brine loop may comprise a pavement salt dissolving and mix tank 16 that receives pavement salt from a pavement salt hopper 18. The pavement salt dissolving and mix tank 16 may be configured to mix or otherwise prepare a salt solution mix to near NaCl saturation before pumping it to the FO system. As previously described, the FO system 5 is then configured to allow water to pass through a membrane 6 from the glycol loop to the product brine loop.

Once a desired amount of water has been drawn through the membrane 6 to the product brine loop, the product brine loop may be configured to pass the solution to a product brine loop tank 22. A product brine transfer pump 24 in the brine loop may be configured to pump the new solution from either the FO system 5 directly or the product brine loop tank 22 to either a pavement deicing brine facility supply tank 28 or a three-way valve control 26. In implementations comprising a three-way valve control, the three-way valve control 26 may be configured to set dry salt addition rate and final pavement deicing brine strength requirements. In various implementations, any percentage of the solution may be passed from the three-way valve control 26 back to the pavement salt dissolving and mix tank 16, and the remaining percentage of the solution may be passed to the pavement deicing brine facility supply tank 28.

### Specifications, Materials, Manufacture, Assembly

It will be understood that implementations are not limited to the specific components disclosed herein, as virtually any components consistent with the intended operation of an osmotic membrane assisted glycol recovery and pavement deicing brine generation system may be utilized. Accordingly, for example, although particular components and so forth, are disclosed, such components may comprise any shape, size, style, type, model version, class, grade, measurement, concentration, material, weight, quantity, and/or the like consistent with the intended operation of an osmotic membrane assisted glycol recovery and pavement deicing brine generation system. Implementations are not limited to uses of any specific components, provided that the components selected are consistent with the intended operation of an osmotic membrane assisted glycol recovery and pavement deicing brine generation system. Accordingly, the components defining any osmotic membrane assisted glycol recovery and pavement deicing brine generation system may be formed of any of many different types of materials or combinations thereof that can be readily formed into shaped objects provided that the components selected are consistent with the intended operation of an osmotic membrane assisted glycol recovery and pavement deicing brine generation system.

### Use

In an implementation, the osmotic membrane assisted glycol recovery and pavement deicing brine generation system 10 may be utilized at an airport, air base, or other type of air field. The system, however, may alternatively also be utilized in any type of environment where deicing and pavement treating salt solutions are utilized. In places where the description above refers to particular implementations, it should be readily apparent that a number of modifications may be made without departing from the spirit thereof and that these implementations may be alternatively applied. This document is intended to cover such modifications as would fall within the true spirit and scope of the disclosure set forth in this document. The presently disclosed implementations are, therefore, to be considered in all respects as illustrative and not restrictive.

1. A system for recycling spent glycol-containing fluid, comprising:
(a) a sump for collecting the fluid;
(b) a filter connected to the sump;
(c) a separation tank connected at its first inlet to the filter, the separation tank having a second inlet and an outlet;
(d) a forward osmosis element having a first inlet and a first outlet, and a second inlet and a second outlet, the first inlet of the forward osmosis element connected to the outlet of the separation tank;
(e) a reclaimed brine tank connected to the first outlet of the forward osmosis element;
(f) a brine loop connecting the first outlet of the forward osmosis element to the second inlet of the forward osmosis element;
(g) a first valve disposed between and connected to the brine loop and the reclaimed brine tank;
(h) a recovered glycol tank; and
(i) a glycol loop connecting the second outlet of the forward osmosis element to the recovered glycol tank and the separation tank, the glycol loop having a second valve disposed between an connected to the glycol tank and the separation tank.

2. The system of claim 1, wherein the glycol-containing fluid is spent aircraft deicing fluid.

3. The system of claim 2, wherein the first valve is a control valve for adding salt to the fluid in the system.

4. A method for recycling a water and glycol-containing fluid, comprising substantially simultaneously extracting and treating water from the fluid by subjecting the fluid to forward osmosis.

5. The method of claim 4, wherein the fluid is spent airplane deicing fluid.

6. A method for reclaiming spent glycol-containing fluid, comprising the following steps:
(a) filtering the fluid;
(b) subjecting the fluid to forward osmosis in a forward osmosis element, to produce a first stream comprising glycol and a second stream comprising primarily brine; and
(c) transporting at least a portion of the second stream to the forward osmosis element's permeate side, to drive forward osmosis in step (b).

7. The method of claim 6 further comprising a step of recovering at least a portion of the first stream comprising glycol.

8. The method of claim 6 further comprising a step of combining at least a portion of the first stream produced in step (b) with additional spent glycol-containing fluid to produce a combined fluid, and subjecting the combined fluid to forward osmosis in a forward osmosis element.

9. The method of claim 6 further comprising recovering at least a portion of the second stream comprising brine.

10. The method of claim 6, wherein step (b) further comprises mixing the second stream with salt.

11. The method of claim 10, further comprising adjusting salt levels introduced into the fluid to control the rate of the method.

12. The method of claim 10, further comprising transporting at least a portion of the second stream to the forward osmosis element's permeate side, to drive forward osmosis in steps (b) and (c).

13. The method of claim 8, wherein the glycol-containing fluid is spent aircraft deicing fluid.

14. A continuous method for reclaiming spent aircraft deicing fluid, comprising the following steps:
(a) filtering the fluid;
(b) subjecting the fluid to forward osmosis in a forward osmosis element, to produce a first stream comprising primarily glycol and a second stream comprising primarily brine;
(c) combining a first portion of the first stream produced in step (b) with additional spent aircraft deicing fluid to produce a combined fluid, and subjecting the combined fluid to forward osmosis in the forward osmosis element;
(d) recovering a second portion of the first stream produced in step (b) comprising primarily glycol;
(e) combining a first portion of the second stream produced in step (b) with salt to produce a brine, and transporting the first portion to the forward osmosis element's permeate side, to drive forward osmosis in steps (b) and (c); and
(f) recovering a second portion of the second stream produced in step (b).

15. A method for reclaiming spent aircraft deicing fluid, comprising the following steps:
(a) filtering the fluid;
(b) subjecting the fluid to forward osmosis in a forward osmosis element, to produce a first stream comprising primarily glycol and a second stream comprising primarily brine;
(c) recovering substantially all of the first stream produced in step (b);
(d) combining a first portion of the second stream produced in step (b) with salt to produce a brine, and transporting the first portion to the forward osmosis element's permeate side, to drive forward osmosis in step (b); and
(e) recovering a second portion of the second stream produced in step (b).
